(19) United States
(12) Patent Application Publication
(10) Pub. No.: US 2003/0162729 A1
(43) Pub. Date: Aug. 28, 2003

(54) Macrolides with activity against methicillin-resistant staphylococcus aureus

(76) Inventors: Zhenkun Ma, Gurnee, IL (US); Stevan Djuric, Libertyville, IL (US); Robert Keyes, Pleasant Prairie, WI (US); Hong Yong, Grayslake, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/075,012

(22) Filed: Feb. 13, 2002

(51) Int. Cl. A61K 31/7052; C07H 17/08

(52) U.S. Cl. 514/28; 536/7.4

(57) ABSTRACT

Compounds having activity against methicillin-resistant staphylococcus aureus (MRSA), the compounds having formula (I)

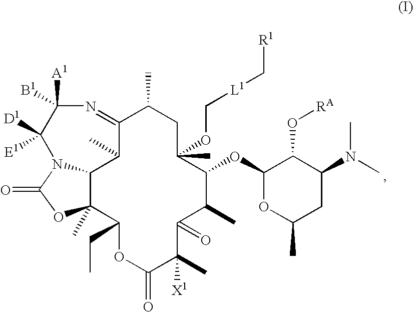

and salts, prodrugs, and salts of prodrugs thereof, processes for making the compounds and intermediates used in the processes, compositions containing the compounds, and methods for prophylaxis and treatment of MRSA infections using the compounds are disclosed.

### TECHNICAL FIELD

This invention is directed to compounds having activity against methicillin-resistant staphylococcus aureus (MRSA), processes for making the compounds and intermediates used in the processes, compositions containing the compounds, and methods for prophylaxis and treatment of MRSA infections using the compounds.

### BACKGROUND OF THE INVENTION

Because the effectiveness of drugs currently available for the prophylaxis and treatment of methicillin-resistant staphylococcus aureus (MRSA) infections is being compromised by increasing bacterial resistance, the development of compounds which demonstrate modified or improved profiles of activity against MRSA would provide significant therapeutic value and an important contribution to the antibacterial arts.

Reference is made to commonly-owned U.S. Pat. No. 6,054,435 which discloses a series of antibacterial compounds but does not teach that the compounds would be useful against MRSA.

### SUMMARY OF THE INVENTION

Accordingly, a first embodiment of the invention is directed to compounds, and salts, prodrugs, and salts of prodrugs thereof, which have surprising activity against MRSA, the compounds being gererically embraced in '1435 and having formula (I)

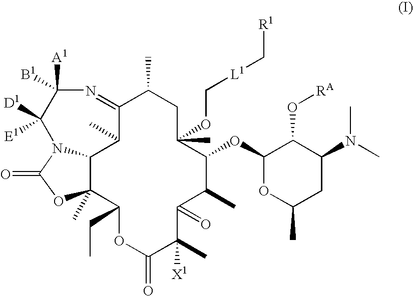

in which,

two of A¹, B¹, D¹, and E¹ are hydrogen, alkyl, alkenyl, alkynyl, cycloalkyl, aryl, heteroaryl, heterocyclyl, —CN, —OH, —SH, —C(O)H, —C(O)R², —C(O)OH, —C(O)OR², —C(O)NR³R⁴, or alkyl substituted with one, two, or three substituents independently selected from the group consisting of —CN, —OH, —SH, halo, aryl, heteroaryl, heterocyclyl, —OR², —SR², —C(O)H, —C(O)R², —C(O)OH, —C(O)OR², —CH═N—OR², —OC(O)R², —OC(O)OR², —C(O) NR³R⁴, —OC(O) NR³R⁴, —NR³R⁴, —N(R⁵)C(O)H, —N(R⁵)C(O)R², —N(R⁵)C(O)NR³R⁴, —N(R⁵)SO₂R², —OR², —SR², —S(O)R², —SO₂R², and —SO₂NR³R⁴, and the remainder are hydrogen; or

A¹ and D¹, A¹ and E¹, B¹ and D¹, or B¹ and D¹ together are one- to five-membered alkylene or two- to five-membered heteroalkylene, and the remainder are hydrogen; or

A¹ and B¹ together are one- to seven-membered alkylene or two- to seven-membered heteroalkylene, and D¹ and E¹ are hydrogen; or

D¹ and E¹ together are one- to seven-membered alkylene or two- to seven-membered heteroalkylene, and A¹ and B¹ are hydrogen;

L¹ is selected from the group consisting of C≡C, (E)—CH═CH, and (Z)—CH═CH;

X¹ is selected from the group consisting of hydrogen and fluoride;

R^{A} is selected from the group consisting of hydrogen and R^{P}, in which R^{P} is a hydroxyl protecting moiety; and

R¹ is selected from the group consisting of aryl, heteroaryl, and heterocycle;

in which, for the foregoing,

each aryl, heteroaryl, and heterocyclyl is unsubstituted or substituted with one, two, three, four, or five substituents independently selected from the group consisting of alkyl, alkenyl, alkynyl, cycloalkyl, halo, —CN, —OH, —SH, —NH₂, —NO₂, (O), —CF₃, —CH₂CF₃, —CF₂CF₃, —OCF₃, —OCH₂CF₃, —OCF₂CF₃, —OR³⁰, —SR³⁰, —S(O)R³⁵, —SO₂R³⁵, —C(O)H, —C(O)R³⁵, —C(O)OH, —C(O)OR³⁵, —NH(R³⁵), —N(R³⁵)(R³⁵′), —C(O)NH₂, —C(O)NH(R³⁵), —C(O)N(R³⁵)(R³⁶), —OC(O)R³⁵, —OC(O)OR³⁵, —OC(O)NH₂, —OC(O)NH(R³⁵), —OC(O)N(R³⁵)(R³⁶), —NHC(O)H, —NHC(O)R³⁵, —NHC(O)OR³⁵, —NHC(O)NH₂, —NHC(O)NH(R³⁵), —NHC(O)N(R³⁵)(R³⁶), —SO₂NH₂, —SO₂NH(R³⁵), —SO₂N(R³⁵)(R³⁶), R⁴⁰, and alkyl substituted with one or two substituents independently selected from the group consisting of halo, —CN, —OH, —SH, (O) —OR³⁰, —SR³⁰, —C(O)OH, —C(O)OR³⁵, —NH₂, —NH(R³⁵), —N(R³⁵)(R³⁶), —C(O)NH₂, —C(O)NH(R³⁵), —C(O)N(R³⁵)(R³⁶), —OC(O)R³⁵, —OC(O)NH₂, —OC(O)NH(R³⁵), —OC(O)N(R³⁵)(R³⁶), —SO₂NH₂, —SO₂NH(R³⁵), —SO₂N(R³⁵)(R³⁶), and R⁴⁰;

R³⁰ is selected from the group consisting of alkyl and alkyl substituted with a substituent selected from the group consisting of halo and OR⁴⁵;

R³⁵ and R³⁶ are independently selected alkyl;

R⁴⁰ is selected from the group consisting of phenyl, naphthyl, furyl, thienyl, pyrrolyl, oxazolyl, thiazolyl, imidazolyl, pyrazolyl, isoxazolyl, isothiazolyl, 1,2,3-oxadiazolyl, 1,2,3-thiadiazolyl, 1,3,4-thiadiazolyl, 1,2,3-triazolyl, tetrazolyl, pyridyl, pyrazinyl, pyrimidinyl, pyrrolidinyl, inidazolidinyl, piperidinyl, piperazinyl, morpholinyl, or thiomorpholinyl, each of which is unsubstituted or substituted with one, two, or three substituents independently selected from the group consisting of alkyl, alkenyl, alkynyl, cycloalkyl, halo, —CN, —OH, —SH, —NO₂, (O), —CF₃, —CH₂CF₃, —CF₂CF₃, —OCF₃, —OCH₂CF₃, —OCF₂CF₃, —OR⁴⁵, —SR⁴⁵, —S(O)R⁵⁰, —SO₂R⁵⁰ , —C(O)H, —C(O)R⁵⁰, —C(O)OH, —C(O)OR⁵⁰, —NH₂, —NH(R⁵⁰), —N(R⁵⁰)(R⁵¹), —C(O)NH₂, —C(O)NH(R⁵⁰), —C(O)N(R⁵⁰)(R⁵¹), —OC(O)R⁵⁰, —OC(O)OR⁵⁰, —OC(O)NH₂, —OC(O)NH(R⁵⁰), —OC(O)N(R⁵⁰)(R⁵¹), —NHC(O)H, —NHC(O)R⁵⁰, —NHC(O)OR⁵⁰, —NHC(O)NH₂, —NHC(O)NH(R⁵⁰), —NHC(O)N(R⁵⁰)(R⁵¹), —SO₂NH₂, —SO₂NH(R⁵⁰), and —SO₂N(R⁵⁰)(R⁵¹);

R⁴⁵ is alkyl; and

R⁵⁰ and R⁵¹ are independently selected alkyl.

A second embodiment of the invention is directed to processes for making the compounds.

A third embodiment of the invention is directed to intermediates which are useful in the second embodiment.

A fourth embodiment of the invention is directed to compositions comprising a therapeutically effective amount of the compounds.

A fifth embodiment of the invention is directed to methods for prophylaxis and treatment of methicillin-resistant staphylococcus aureus infections in a fish or a mammal comprising administering thereto a therapeutically effective amount of the compounds.

In a preferred third, fourth, or fifth embodiment of the invention, the beneficiary of prophylaxis or treatment of methicillin-resistant staphylococcus aureus infections is a mammal.

In a more preferred third, fourth, or fifth embodiment of the invention, the beneficiary of prophylaxis or treatment of methicillin-resistant staphylococcus aureus infections is a human.

### DETAILED DESCRIPTION OF THE INVENTION

The compounds of the invention comprise a parent moiety and variable moieties, the latter of which are identified by a capital letter and accompanying numerical superscript, in which

the term “alkenyl” means a monovalent, straight or branched hydrocarbon, having two to eight carbon atoms and at least one carbon-carbon double bond, attached through a carbon atom;

the term “alkynyl” means a monovalent, straight or branched hydrocarbon, having two to eight carbon atoms and at least one carbon-carbon triple bond, attached through a carbon atom;

the term “alkyl” means a monovalent, saturated, straight or branched hydrocarbon, having one to eight carbon atoms, attached through a carbon atom;

the term “alkylene” means a divalent, saturated, straight or branched hydrocarbon, having one to eight carbon atoms, attached through carbon atoms;

the term “aryl” means monovalent phenyl, attached through a carbon atom, unfused or fused with cycloalkyl, cycloalkenyl, heteroaryl, another phenyl, naphthyl, or the saturated part of indan;

the term “cycloalkyl” means a monovalent, saturated cyclic hydrocarbon, having three to eight carbon atoms, attached through a carbon atom;

the term “halo” means fluoro (—F), chloro (—Cl), or bromo (—Br), and iodo (—I);

the term “heteroaryl” means a monovalent, aromatic, five-membered ring having two double bonds and one oxygen or one sulfur atom, one, two, three, or four nitrogen atoms, or one or two nitrogen atoms and one oxygen or one sulfur atom and the remaining atoms are carbon atoms, attached through a carbon or nitrogen atom, and unfused or fused with phenyl, cycloalkyl, cycloalkenyl, heterocycle, or another heteroaryl; and a monovalent aromatic, six-membered ring having three double bonds and one, two, or three nitrogen atoms and the remaining atoms are carbon atoms, attached through a carbon atom and unfused or fused with phenyl, cycloalkyl, cycloalkenyl, heterocycle, or another heteroaryl; and

the term “heterocyclyl” means a monovalent, non-aromatic three- or four-membered ring having one nitrogen, oxygen, or sulfur atom and the remaining atoms are carbon atoms, zero double bonds, attached through a carbon or nitrogen atom and unfused or fused with phenyl or heteroaryl; a monovalent, non-aromatic five-membered ring having one or two nitrogen, oxygen, or sulfur atoms, and the remaining atoms are carbon atoms, and zero or one double bonds, attached through a carbon or nitrogen atom and unfused or fused with phenyl or heteroaryl; and a monovalent, non-aromatic six or seven-membered ring having one, two, or three nitrogen, oxygen, or sulfur atoms and the remaining atoms are carbon atoms, and zero, one, or two double bonds, attached through a carbon or nitrogen atom and unfused or fused with phenyl or heteroaryl.

Preferred A¹, B¹, D¹, and E¹ moieties are hydrogen.

A preferred L¹ moiety is C≡C.

A preferred X¹ moiety is hydrogen.

A preferred R^{A} moiety is hydrogen.

Preferred R¹ moieties are 4-(furan-2-yl)phenyl, 4-(2-methyl-2H-tetraazol-5-yl)phenyl, pyrid-2-yl, 4-(pyridin-2-yl)phenyl, quinolin-3-yl, 4-(1,2,3-thiadiazol-5-yl)phenyl, 4-(1,3-thiazol-2-yl)phenyl, 4-(thien-2-yl)phenyl, and 4-(vinyl)phenyl.

These preferred variable moieties combine with the parent moiety to form a preferred first embodiment of the invention, the preferred first embodiment comprising compounds, and salts, prodrugs, and salts of prodrugs thereof, having formula (I)

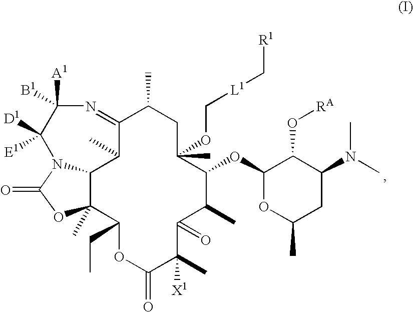

in which

A¹ B¹, D¹, and E¹ are hydrogen;

X¹ is hydrogen;

L¹ is C≡C;

R^{a} is hydrogen;

R¹ is selected from the group consisting of aryl, heteroaryl,

in which the aryl is phenyl and the heteroaryl is pyridyl and quinolinyl, and

in which the foregoing aryl and each foregoing heteroaryl is unsubstituted or substituted with a substituent selected from the group consisting of alkenyl and R⁴⁰,

in which R⁴⁰ is selected from the group consisting of furyl, pyridyl, 1,2,3-thiadiazolyl, thiazolyl, thienyl, and tetrazolyl, each of which is unsubstituted or substituted with one alkyl substituent.

These preferred variable moieties also combine to form another preferred first embodiment of the invention, the preferred first embodiment comprising compounds, and salts, prodrugs, and salts of prodrugs thereof, having formula (I)

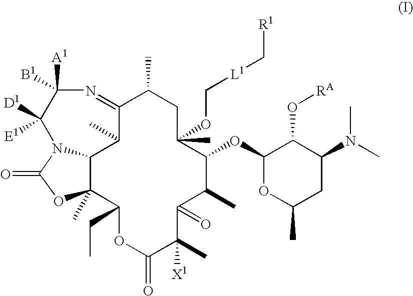

in which

A¹, B¹, D¹, and E¹ are hydrogen;

X¹ is hydrogen;

L¹ is C≡C;

R^{A} is hydrogen;

R¹ is selected from the group consisting of aryl and heteroaryl,

in which the aryl is phenyl and the heteroaryl is pyridyl and quinolinyl, and

in which the foregoing aryl and each foregoing heteroaryl is unsubstituted or substituted with a substituent selected from the group consisting of C₂-alkenyl and R⁴⁰,

in which R⁴⁰ is selected from the group consisting of furyl, pyridyl, 1,2,3-thiadiazolyl, thiazolyl, thienyl, and tetrazolyl, each of which is unsubstituted or substituted with one C₁-alkyl substituent.

These preferred variable moieties also combine to form still yet another preferred first embodiment of the invention, the preferred first embodiment comprising compounds, and salts, prodrugs, and salts of prodrugs thereof, which are selected from the group consisting of

(3aS,4R,7R,9R,10R,11S,13R,15R,15aR)-4-ethyl-3a,7,9,11,13,15-hexamethyl-2,6,8-trioxo-11-((4-pyridin-2-ylbut-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside,

(3aS,4R,7R,9R,10R,11S,13R,15R,15aR)-4-ethyl-3a,7,9,11,13,15-hexamethyl-2,6,8-trioxo-11-((4-(4-(1,2,3-thiadiazol-5-yl)phenyl)but-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside,

(3aS,4R,7R,9R,10R,11S,13R,15R,15aR)-4-ethyl-3a,7,9,11,13,15-hexamethyl-2,6,8-trioxo-11-((4-quinolin-3-ylbut-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside,

(3aS,4R,7R,9R,10R,11S,13R,15R,15aR)-4-ethyl-3a,7,9,11,13,15-hexamethyl-2,6,8-trioxo-11-((4-(4-thien-2-ylphenyl)but-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside,

(3aS,4R,7R,9R,10R,11S,13R,15R,15aR)-4-ethyl-3a,7,9,11,13,15-hexamethyl-2,6,8-trioxo-11-((4-(4-(1,3-thiazol-2-yl)phenyl)but-2-ynyl)oxy)dodecahydro (epiazenoethano)oxacyclotetradecino[4,3-d][1,3]oxazol-10-yl 3,4,6-trideoxy-3-(dimethylamino)-β-D-xylo-hexopyranoside,

(3aS,4R,7R,9R,10R,11S,13R,15R,15aR)-4-ethyl-11-((4-(4-(2-furyl)phenyl)but-2-ynyl)oxy)-3a,7,9,11,13,15-hexamethyl-2,6,8-trioxododecahydro-14,1-(epiazenoethano)oxacyclotetradecino[4,3-d][1,3]oxazol-10-yl 3,4,6-trideoxy-3-(dimethylamino)-β-D-xylo-hexopyranoside,

(3aS,4R,7R,9R,10R,11S,13R,15R,15aR)-4-ethyl-3a,7,9,11,13,15-hexamethyl-2,6,8-trioxo-11-((4-(4-vinylphenyl)but-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside,

(3aS,4R,7R,9R,10R,11S,13R,15R,15aR)-4-ethyl-3a,7,9,11,13,15-hexamethyl-2,6,8-trioxo-11-((4-(4-pyridin-2-ylphenyl)but-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside, and

(3aS,4R,7R,9R,10R,11S,13R,15R,15aR)-4-ethyl-3a,7,9,11,13,15-hexamethyl-11-((4-(4-(2-methyl-2H-tetraazol-5-yl)phenyl)but-2-ynyl)oxy)-2,6,8-trioxododecahydro-14,1-(epiazenoethano)oxacyclotetradecino[4,3-d][1,3]oxazol-10-yl 3,4,6-trideoxy-3-(dimethylamino)-β-D-xylo-hexopyranoside.

The compounds of the invention comprise asymmetrically substituted carbon atoms in the R or S configuration. Asymmetric carbon atoms with equimolar amounts of R and S configurations are racemic. Atoms with an excess of one configuration over the other are assigned the configuration in the higher amount, preferably an excess of about 85%-90%, more preferably an excess of about 95%-99%, and still more preferably an excess greater than about 99w.

The terms “R” and “IS” are as defined by the IUPAC 1974 Recommendations for Section E, Fundamental Stereochemistry, Pure Appl. Chem. (1976) 45, 13-10.

Accordingly, all stereoisomers of the compounds of the invention, including racemic mixtures, mixtures of diastereomers, and single diastereomers, are meant to be embraced by the invention.

The compounds of the invention may also comprise carbon-carbon double bonds as being in the Z or E configuration, in which the term “Z” represents the larger two of the four substituents disposed on same side of a carbon-carbon double bond and the term “E” represents the larger two of the four substituents disposed on opposite sides of a carbon-carbon double bond. The compounds may also exist as an equilibrium mixture comprising Z or E configurations.

The compounds of the invention containing hydroxyl, amino, or carboxylic acids may have attached thereto prodrug-forming moieties. The prodrug-forming moieties are removed by metabolic processes and release the compounds having the freed hydroxyl, amino, or carboxylic acid in vivo. Prodrugs are useful for adjusting such pharmacokinetic properties of the compounds as solubility and/or hydrophobicity, absorption in the gastrointestinal tract, bioavailability, tissue penetration, and rate of clearance.

The compounds of the invention may be prepared by synthetic processes or metabolic processes. Metabolic processes include those processes occurring in vitro and in vivo.

The compounds of the invention may exist as acid addition salts, basic addition salts, or zwitterions. Salts of the compounds are prepared during their isolation or following their purification. Acid addition salts of the compounds are those derived from the reaction of the compounds with an acid. For example, the acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, formate, fumarate, hydrochloride, hydrobromide, hydroiodide, lactate, maleate, mesitylenesulfonate, methanesulfonate, naphthylenesulfonate, nicotinate, oxalate, pamoate, pectinate, persulfate, picrate, propionate, succinate, tartrate, thiocyanate, trichloroacetic, trifluoroacetic, phosphate, glutamate, bicarbonate, para-toluenesulfonate, lactobionate, and undecanoate salts of the compounds and prodrugs thereof are contemplated as being within the scope of the invention. When the compounds contain carboxylic acids, basic addition salts may be prepared therefrom by reaction with a base such as the hydroxide, carbonate, or bicarbonate of cations such as lithium, sodium, potassium, calcium, and magnesium.

The compounds of the invention may be administered with or without an excipient. Excipients include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof. Excipients for orally administered compounds in solid dosage forms include agar, alginic acid, cocoa butter, gelatin, isotonic saline, malt, powdered tragacanth, Ringer's solution, talc, water, aluminum hydroxide, magnesium hydroxide, sodium and potassium phosphate salts, cellulose, cellulose acetate, ethyl cellulose, sodium carboxymethyl cellulose, ethyl laureate, ethyl oleate, magnesium stearate, sodium lauryl sulfate, castor oil, corn oil, cottonseed oil, germ oil, groundnut oil, olive oil, peanut oil, safflower oil, sesame oil, soybean oil, benzyl alcohol, benzyl benzoate, 1,3-butylene glycol, ethanol, ethyl acetate, ethyl carbonate, glycerol, isopropanol, propylene glycol, tetrahydrofurfuryl alcohol, corn starch, potato starch, lactose, glucose sucrose, and mixtures thereof. Excipients for ophthalmically and orally administered compounds in liquid dosage forms include water, ethanol, isopropanol, ethyl carbonate, ethyl acetate, benzyl alcohol, benzyl benzoate, propylene glycol, 1,3-butylene glycol, cottonseed oil, groundnut oil, corn oil, germ oil, olive oil, castor oil, sesame oil, glycerol, tetrahydrofurfuryl alcohol, polyethylene glycols, fatty acid esters of sorbitan, and mixtures thereof. Excipients for osmotically administered compounds include water, ethanol, isopropanol, chlorofluorohydrocarbons, and mixtures thereof. Excipients for parenterally administered compounds include water, 1,3-butanediol, Ringer's solution, U.S.P. or isotonic sodium chloride solution, oleic acid, castor oil, corn oil, cottonseed oil, germ oil, groundnut oil, olive oil, peanut oil, safflower oil, sesame oil, soybean oil, liposomes, and mixtures thereof. Excipients for rectally and vaginally administered compounds include cocoa butter, polyethylene glycol, wax, and mixtures thereof.

The compounds of the invention may be administered parenterally (subcutaneously, intravenously, intramuscularly, and intrasternally), orally, osmotically, ophthalmically, rectally, topically, and vaginally. Orally administered compounds in solid dosage forms may be administered as capsules, dragees, granules, pills, powders, and tablets. Ophthalmically and orally administered compounds in liquid dosage forms may be administered as elixirs, emulsions, microemulsions, solutions, suspensions, and syrups. Osmotically and topically administered compounds may be administered as creams, gels, inhalants, lotions, ointments, pastes, powders, solutions, and sprays. Parenterally administered compounds may be administered as aqueous or oleaginous solutions or aqueous or oleaginous suspensions, the latter of which contains crystalline, amorphous, or otherwise insoluble forms of the compounds. Rectally and vaginally administered compounds may be administered as creams, gels, lotions, ointments, and pastes.

Dosage forms for the compounds of the invention depend on the species being treated, the disorder being treated and the severity thereof, the composition comprising the compounds, the time of administration, the route of administration, the duration of treatment, the potency of the compounds, and the rate of excretion of the compounds. The daily therapeutically effective amount of the compounds administered to a patient in single or divided doses range from about 0.1 to about 200 mg/kg body weight, preferably from about 0.25 to about 100 mg/kg body weight. Single dose compositions contain these amounts of the compounds or combinations of submultiples thereof.

To determine antibacterial activity of the compounds of the invention, twelve petri dishes, each containing successive aqueous dilutions of test compounds in sterilized Brain Heart Infusion agar (Difco 0418-01-5) (10 mL), were inoculated with 1:100 dilutions of MRSA 1775 using a Steers replicator block (or 1:10 dilutions for slow-growing Streptococcus strains), co-incubated at 35-37° C. for 20-24 hours with a plate with an erythromycin A standard and a control plate with no compound, and inspected visually to provide the minimum inhibitory concentration (MIC), in μg/mL, by which is meant the lowest concentration of the test compound which yielded no growth, a slight haze, or sparsely isolated colonies on the inoculum spot as compared to growth in the control plate.

All of the compounds tested displayed activity against MRSA superior to their respective controls. In a preferred range, the compounds demonstrated MIC's in a range of about 2 μg/mL to about 64 μg/mL; and in a more preferred range, the compounds demonstrated MIC's in a range of about 2 μg/mL to about 8 μg/mL.

The compounds are therefore useful as antibacterials against MRSA.

The following schemes illustrate representative processes by which the compounds of the invention may be prepared with the understanding that the order of the steps in the processes may be varied, other reagents may be substituted for those specifically mentioned, and vulnerable substituents may be protected and deprotected during the process.

Abbreviations used are: DME for 1,2-dimethoxyethane; DMF for N,N-dimethylformamide; and THF for tetrahydrofuran.

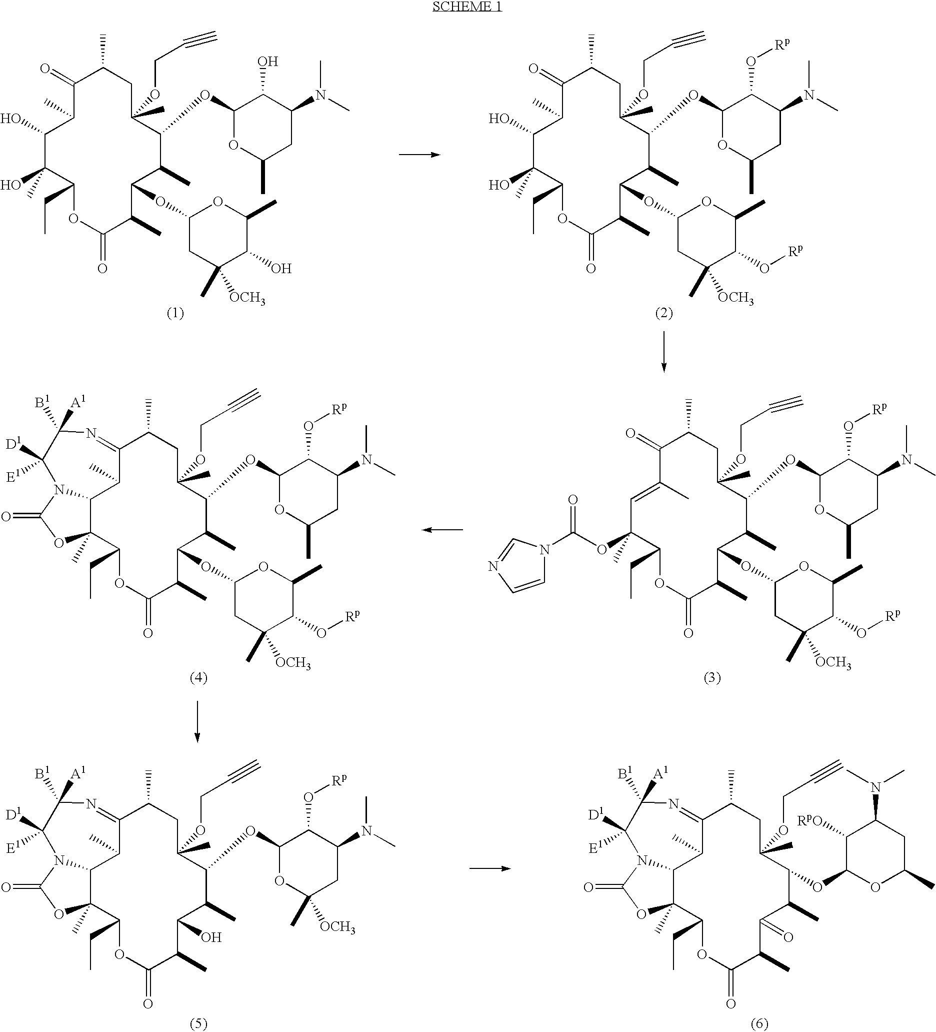

Compounds having formula (1) may be converted to compounds having formula (2), in which R^{P} is acetyl (CH₃C(O)), benzoyl (C₆H₅C(O)), or trimethylsilyl, by reacting the former, a hydroxyl protecting reagent, a first base, and, optionally, N,N-dimethylaminopyridine. Hydroxyl protecting reagents include acetic anhydride, acetyl chloride, benzoic anhydride, benzoyl chloride, and trimethylsilyl chloride. First bases include triethylamine, diisopropylethylamine, pyridine, and lutidine. The reaction is typically conducted at about 0° C. to 60° C., over about 4 to 24 hours, in solvents such as dichloromethane, chloroform, THF, DME, and tert-butyl methylether.

Compounds having formula (2) may be converted to compounds having formula (3) by reacting the former, carbonyldiimidazole, a second base, and, optionally, N,N-dimethylaminopyridine. Second bases include 1,8-diazabicyclo-[5.4.0]undec-7-ene, lithium bis(trimethylsilyl)amide, sodium bis(trimethylsilyl)amide, and potassium bis(trimethylsilyl)amide. The reaction is typically conducted at about 25° C., over about 6 to 24 hours, in solvents such as THF, DMF, 1,4-dioxane, and N-methylpyrrolidine.

Compounds having formula (3) may be converted to compounds having formula (4) by (a) reacting the former and a compound having formula (i)

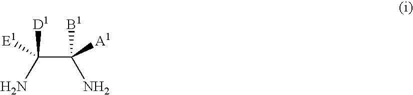

and (b) reacting the product of step (a) with a dilute first acid. First acids include hydrochloric acid, triflic acid, para-toluenesulfonic acid, and trifluoroacetic acid. Step (a) is typically conducted at about 25° C., over about 24 hours to 72 hours, in solvents such as acetonitrile, DMF, water, and mixtures thereof. Step (b) is typically conducted at about 70° C. to 100° C., over about 12 hours to about 24 hours, in solvents such as benzene, toluene, xylene, and mixtures thereof.

Compounds having formula (4) may be converted to compounds having formula (5) by reacting the former and a second acid. Second acids include hydrochloric acid, triflic acid, para-toluenesulfonic acid, and trifluoroacetic acid. The reaction is typically conducted at about 60° C., over about 12 to 24 hours, in solvents such as ethanol, acetone, THF, water, and mixtures thereof.

Compounds having formula (5) may be converted to compounds having formula (6) by reacting the former, a first oxidizing agent, and, optionally, a first additive. First oxidizing agents include dimethylsulfide/N-chlorosuccinimide, dimethylsulfoxide/l-(3-dimethylaminopropyl)-3-ethylcarbodiimide, and dimethylsulfoxide/oxalyl chloride. First additives include phosphoric acid, pyridinium trifluoroacetate, silica gel, triethylamine, and pyridine. The reaction is typically conducted at about −10° C. to 25° C., over about 3 to 24 hours, in solvents such as THF, DMSO, and dichloromethane.

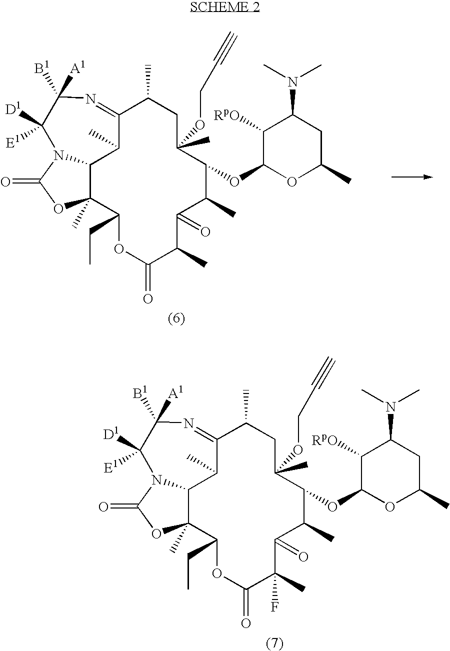

Compounds having formula (6) may be converted to compounds of formula (7) by reacting the former, a fluorinating agent and, optionally, a second base. Fluorinating agents include 3,5-dichloro-1-fluoropyridinium tetrafluoroborate, N-fluorobenzenesulfonimide, 3,5-dichloro-1-fluoropyridinium triflate, N-fluoro-N-methyl-para-toluenesulfonamide, N-fluoropyridinium triflate, and N-fluoroperfluoropiperidine. Second bases include sodium hydride, potassium hydride, lithium diisopropylamide, triethylamine, and N,N-diisopropylethylamine. The reaction is typically conducted at about −78° C. to 0° C., over about 2 to 24 hours, in solvents such as DMF, THF, diethyl ether, and mixtures thereof.

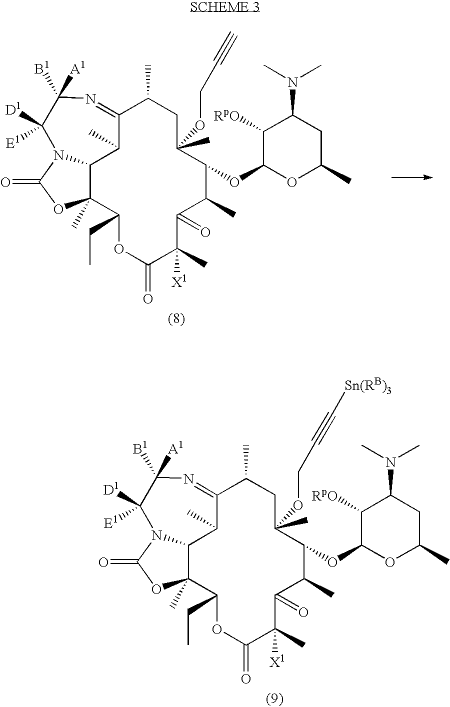

Compounds having formula (8) may be converted to compounds having formula (9) by reacting the former, a stannylating agent and, optionally, a coupling catalyst. Stannylating agents include tributyltin ethoxide, tributyltin methoxide, hexamethyldistannane, and hexabutyldistannane. Coupling catalysts include tetrakis(triphenylphosphine)palladium(0), and tris(dibenzylideneacetone)dipalladium(0). The reaction is typically conducted neat at about 80° C. to 150° C., over about 8 to 48 hours, or in solvents such as toluene, xylenes, 1,4-dioxane, and THF.

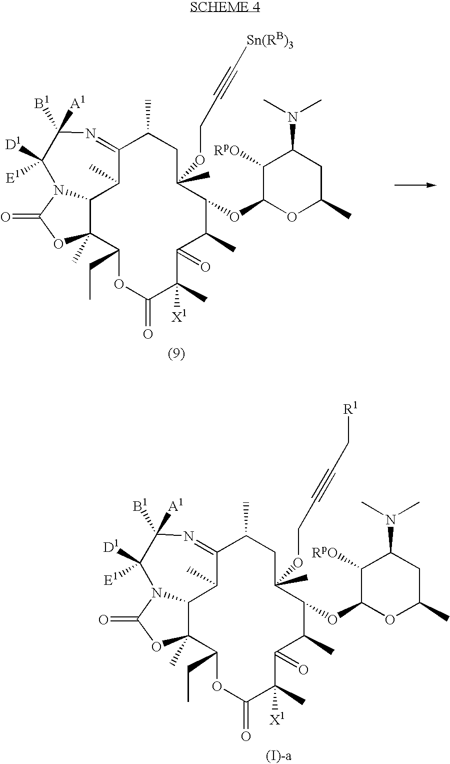

Compounds having formula (9) may be converted to compounds having formula (I)-a by reacting the former, a compound having formula (ii)

X²—CH₂—R¹ (ii),

in which X² is Cl, Br, or I, a coupling catalyst, and a third base. Coupling catalysts include tetrakis(triphenylphosphine)palladium(0), tris(dibenzylideneacetone)dipalladium(0), and dichlorobis(triphenylphosphine)palladium(II). Third bases include sodium carbonate, sodium bicarbonate, potassium carbonate, cesium carbonate, triethylamine, and 10 diisopropylethylamine. The reaction is typically conducted in a sealed vessel at about 80° C. to 150° C., over about 2 to 24 hours, or in solvents such as toluene, xylenes, 1,4-dioxane, and THF.

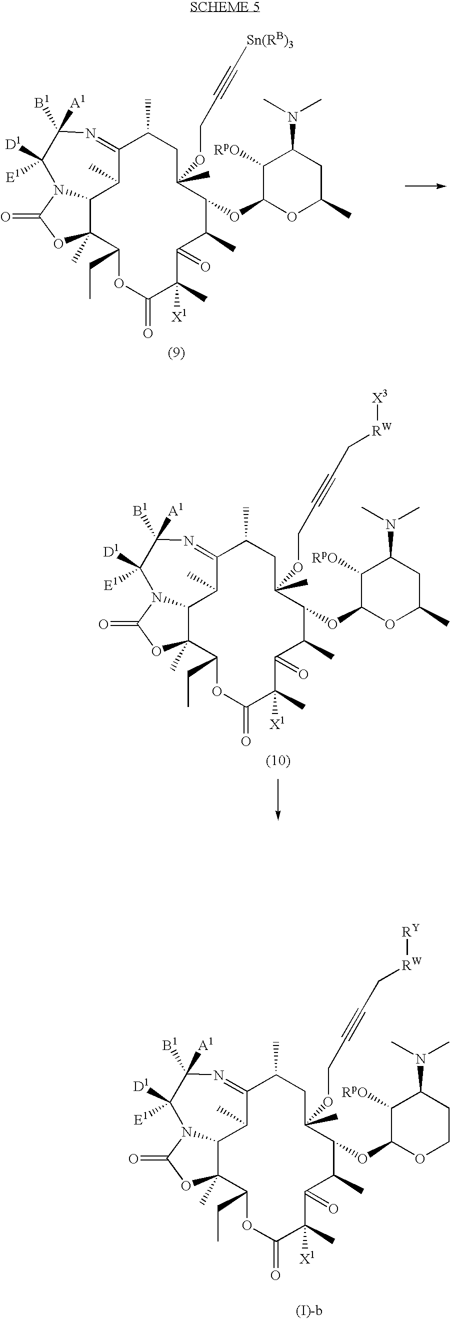

Compounds having formula (9) may be converted to compounds having formula (10) by reacting the former, compounds having formula (iii)

X²—R^{W}—X³ (iii),

in which X³ is Cl, Br, or I and

R^{W} is aryl or heteroaryl, under the same conditions described for the conversion of compounds having formula (9) to compounds having formula (I)-a in SCHEME 4.

Compounds having formula (10) may be converted to compounds having formula (I)-a by reacting the former, a compound having formula (iv)

Q¹—R^{Y} (iv)

in which Q¹ is B(V¹)₂ or Sn(R^{B})₃,

each V¹ is independently hydrogen, alkyl, —OH, or —OR⁴⁵, and

R^{Y} and R^{W} combine to form the moieties embraced by R¹, under the same conditions described for the conversion of compounds having formula (9) to compounds having formula (I)-a in SCHEME 4.

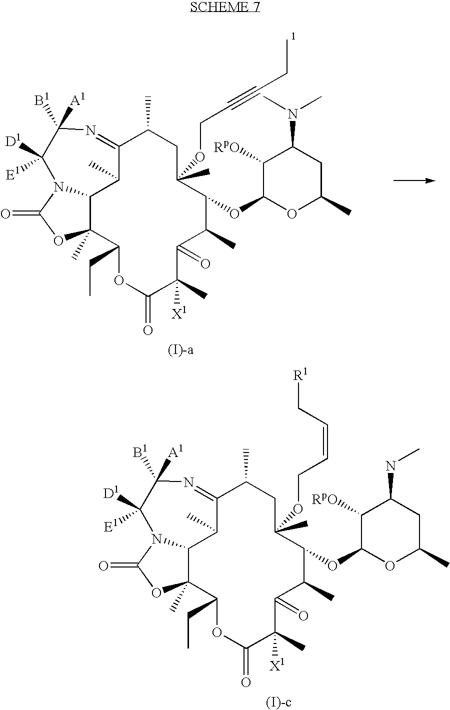

Compounds having formula (I)-a may be converted to compounds having formula (I)-c by reacting the former, hydrogen gas, a hydrogenation catalyst, and, optionally, quinoline. Hydrogenation catalysts include Lindlar catalyst and palladium on barium sulfate. The reaction is typically conducted at 25° C., over about 1 to 6 hours, in solvents such as methanol, ethanol, propanol, butanol, iso-propanol, tert-butanol, acetonitrile, THF, ethyl acetate, and mixtures thereof.

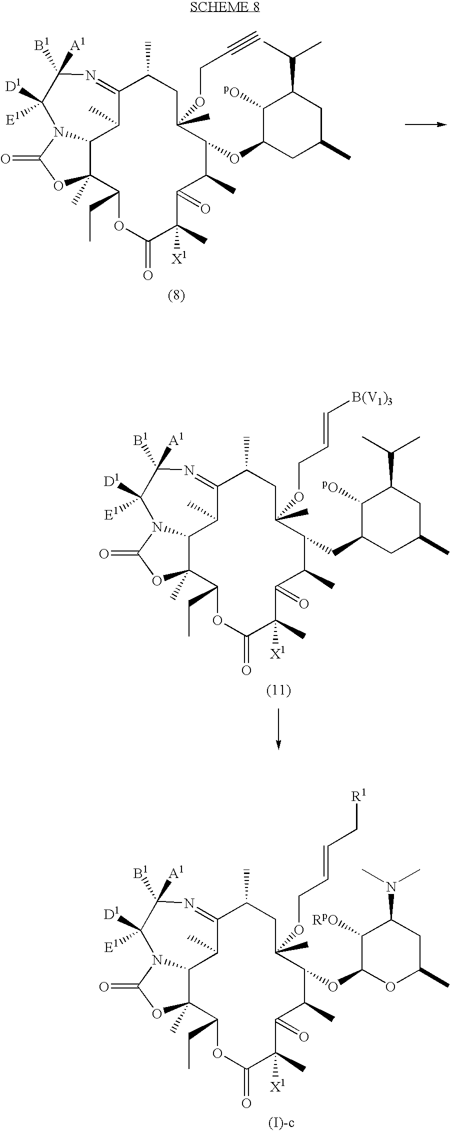

Compounds having formula (8) may be converted to compounds having formula (11) by reacting the former and compounds having formula (v)

B(V¹)₃, (v).

The reaction is typically conducted at about −20° C. to 25° C., over about 1 to 6 hours, in solvents such as THF, DME, and diethyl ether.

Compounds having formula (11) may be converted to compounds having formula (I)-c by reacting the former and compounds having formula (vi)

X¹—R¹ (vi),

under the same conditions described for the conversion of compounds having formula (9) to compounds having formula (I)-a in SCHEME 4.

Compounds having formula (I), in which R^{A} is R^{P}, and R^{P} is acetyl or benzoyl, may be converted to compounds having formula (I), in which R^{A} is hydrogen, by reacting the former and methanol. The reaction is typically conducted at about 25° C. to 65° C., over about 2 to 60 hours, in methanol.

Compounds having formula (I), in which R^{A} is R^{P}, and R^{P} is trimethylsilyl, may be converted to compounds having formula (I), in which R^{A} is hydrogen, by reacting the former and a fluoride-donating agent. Fluoride-donating agents include tetrabutylammonium fluoride, polymer-bound ammonium fluoride, tetrabutylammonium fluoride, pyridine.HF, and triethylamine-trihydrofluoride. The reaction is typically conducted at about 0° C. to 50° C., over about 1 to 24 hours, in solvents such as THF and 1,4-dioxane.

The following examples illustrate methods by which certain preferred first embodiments of the invention may be prepared.

### EXAMPLE 1

This example was prepared as described in commonly owned U.S. Pat. No. 6,075,133, EXAMPLE 246, step 246c.

### EXAMPLE 2

A solution of EXAMPLE 1 (10 g), N′N-dimethylaminopyridine (50 mg), and triethylamine (3.8 mL) in dichloromethane (70 mL) at 15° C. was treated with benzoic anhydride (7.02 g) over 10 minutes, stirred for 20 minutes, warmed to ambient temperature, stirred for 7 hours, diluted with ethyl acetate, washed with 5% sodium carbonate, water, and brine, and dried (Na₂SO₄), filtered, and concentrated.

### EXAMPLE 3

A solution of EXAMPLE 2 (9.8 g), carbonyldiimidazole (4.05 g), N′N-dimethylaminopyridine (122 mg), and 1,8-diazabicyclo[5.4.0]undec-7-ene (2.24 mL) in THF (45 mL) and DMF (13 mL) was stirred for 12 hours, diluted with ethyl acetate, washed with water and brine, and dried (Na₂SO₄), filtered, and concentrated.

### EXAMPLE 4

A solution of EXAMPLE 3 (11.09 g) and ethylenediamine (6.67 mL) in acetonitrile (50 mL) and water (5 mL) at ambient temperature was stirred for 3 days and concentrated. The concentrate was dissolved in toluene (140 mL) and acetic acid (7 mL), and this solution was heated at 80° C. for 12 hours then cooled, stirred for 12 hours, diluted with dichloromethane, washed with saturated potassium carbonate, and dried (Na₂SO₄), filtered, and concentrated; and the concentrate was flash chromatographed on silica gel with 95:5:0.5 dichloromethane/methanol/concentrated ammonium hydroxide.

### EXAMPLE 5

A solution of EXAMPLE 4 (5.95 g) and 2M HCl (5 mL) in ethanol (5 mL) was stirred at 55° C. for 12 hours and concentrated. The concentrate was dissolved in water, and this solution was washed with diethyl ether, treated with concentrated ammonium hydroxide, and extracted with dichloromethane; and the extract was concentrated.

### EXAMPLE 6

A solution of N-chlorosuccinimide (5.46 g) in dichloromethane (200 mL) at −10° C. was treated with dimethyl sulfide (3.50 mL), stirred for 10 minutes, treated with a solution of EXAMPLE 5 (20.9 g) in dichloromethane (90 mL) over 30 minutes, stirred for 60 minutes, treated with triethylamine (3.79 ml), stirred for 90 minutes, washed with 5% sodium bicarbonate and brine, and dried (Na₂SO₄), filtered, and concentrated.

### EXAMPLE 7

A solution of EXAMPLE 6 (3.825 g) and tributyltin ethoxide (1.76 mL) was heated at 110° C. for 48 hours, with an additional tributyltin ethoxide (1.76 mL) treatment after 24 hours, and concentrated; and the concentrate was dissolved in acetonitrile and treated with hexane.

### EXAMPLE 8

A solution of EXAMPLE 7 (2.11 g), 1-bromo-4-(bromomethyl)benzene (797 mg), and tetrakis(triphenylphosphine)palladium(0) (116 mg) in toluene (10 mL) was heated at 90° C. in a sealed tube for 3 hours and concentrated; and the concentrate was flash chromatographed on silica gel with 97:3:0.5 dichloromethane/methanol/concentrated ammonium hydroxide.

### EXAMPLE 9

A solution of EXAMPLE 7 (220 mg), 2-bromomethylpyridine (84 mg), and tetrakis(triphenylphosphine)palladium(0) (11 mg) in toluene (2 mL) was heated at 80° C. in a sealed tube for 12 hours, diluted with ethyl acetate, washed with saturated sodium bicarbonate and brine, and dried (Na₂SO₄), filtered, and concentrated; and the concentrate was flash chromatographed on silica gel with 97:3:0.5 dichloromethane/methanol/concentrated ammonium hydroxide.

### EXAMPLE 10

A solution of EXAMPLE 9 in methanol was heated at 65° C. for 3 hours and concentrated; and the concentrate was flash chromatographed on silica gel with 95:5:0.5 dichloromethane/methanol/concentrated ammonium hydroxide.

### EXAMPLE 11

This example was prepared by substituting 5-[4-(bromomethyl)phenyl]-1,2,3-thiadiazole for 2-bromomethylpyridine in EXAMPLES 9 and 10.

### EXAMPLE 12

This example was prepared by substituting 3-bromomethylquinoline for 2-bromomethylpyridine in EXAMPLES 9 and 10.

### EXAMPLE 13

A solution of EXAMPLE 8 (100 mg), 2-(tributylstannyl)thiophene (0.054 mL), and tetrakis(triphenyphosphine)palladium(0) (6.2 mg) in toluene (1 mL) was heated at 80° C. for 12 hours in a sealed tube, diluted with ethyl acetate, washed with saturated sodium bicarbonate and brine, and dried (Na₂SO₄), filtered, and concentrated; and the concentrate was flash chromatographed on silica gel with 1:1 acetone/hexane.

### EXAMPLE 14

A solution of EXAMPLE 13 in methanol at 65° C. was stirred for 3 hours and concentrated; and the concentrate was flash chromatographed on silica gel with 95:5:0.5 dichloromethane/methanol/concentrated ammonium hydroxide.

### EXAMPLE 15

This example was prepared by substituting 2-(tributylstannyl)thiazole for 2-(tributylstannyl)thiophene in EXAMPLES 13 and 14.

### EXAMPLE 16

This example was prepared by substituting 2-(tributylstannyl)furan for 2-(tributylstannyl)thiophene in EXAMPLES 13 and 14.

### EXAMPLE 17

This example was prepared by substituting vinyltributylstannane for 2-(tributylstannyl)thiophene in EXAMPLES 13 and 14.

### EXAMPLE 18

This example was prepared by substituting 2-(tributylstannyl)pyridine for 2-(tributylstannyl)thiophene in EXAMPLES 13 and 14.

### EXAMPLE 19

This example was prepared by substituting 2-methyl-5-(tributylstannyl)-2H-tetrazole for 2-(tributylstannyl)thiophene in EXAMPLES 13 and 14.

### EXAMPLE 20

A solution of EXAMPLE 6 (672 mg) in DMF (5 mL) at 0° C. was treated with 60% oily sodium hydride (70 mg), stirred for 40 minutes, treated with N-fluorobenzenesulphonimide (314 mg), stirred for 3 hours, diluted with ethyl acetate, washed with water and brine, and dried (Na₂SO₄), filtered, and concentrated; and the concentrate was flash chromatographed on silica gel with 95:5:0.5 dichloromethane/methanol/concentrated ammonium hydroxide.

### SPECTRAL DATA FOR REPRESENTATIVE COMPOUNDS

### EXAMPLE 10

¹³C NMR (CDCl₃) δ 204.7, 169.7, 154.6, 152.4, 139.7, 127.0, 118.3, 116.8, 107.4, 101.8, 82.0, 80.4, 80.0, 76.7, 75.9, 70.8, 69.3, 68.2, 66.4, 59.5, 52.0, 50.0, 46.6, 46.1, 40.5, 38.6, 36.5, 29.6, 22.2, 20.9, 20.7, 18.8, 14.7, 14.5, 13.4, 10.4, 8.6.

### EXAMPLE 11

¹³C NMR (CDCl₃) δ 204.9, 169.4, 156.1, 151.0, 138.2, 129.8, 128.8, 127.6, 103.5, 83.3, 81.6, 80.1, 80.0, 70.3, 69.6, 65.9, 60.2, 51.1, 51.0, 49.5, 47.0, 42.8, 42.1, 40.2, 38.2, 36.3, 28.2, 27.8, 27.1, 25.2, 22.5, 21.3, 20.1, 19.7, 15.6, 14.8, 14.6, 13.6, 13.1, 11.1, 10.8.

### EXAMPLE 12

¹³C NMR (CDCl₃) δ 204.9, 169.6, 156.1, 151.0, 134.3, 129.2, 128.9, 127.6, 126.7, 122.9, 103.5, 82.3, 81.6, 81.0, 80.1, 70.3, 69.6, 65.9, 60.0, 51.1, 51.0, 49.5, 47.0, 42.9, 42.1, 40.2, 38.2, 36.3, 28.2, 27.8, 27.1, 23.0, 22.4, 21.2, 20.1, 19.7, 15.6, 14.8, 14.6, 13.6, 13.0, 11.1, 10.7.

### EXAMPLE 14

¹H NMR (CDCl₃) δ 7.55 (2H, d), 7.34 (2H, d), 7.27 (1H, m), 7.07 (2H, m), 4.94 (1H, m), 4.42 (1H, d), 4.33 (1H, d), 3.90-3.75 (6H, m), 3.68 (1H, m), 3.63 (1H, m), 3.54 (1H, m), 3.38 (1H, m), 3.19 (2H, m), 2.81 (2H, m), 2.48 (2H, m), 2.28 (6H, s), 1.96 (2H, m), 1.70-1.58 (5H, m), 1.52 (6H, m), 1.42 (1H, m), 1.38-1.30 (5H, m), 1.23 (11H, m), 1.07 (3H, d), 0.92-0.85 (5H, m).

### EXAMPLE 15

¹H NMR (CDCl₃) δ 7.89 (2H, d), 7.44 (2H, d), 7.38 (1H, s), 7.22 (1H, s), 5.04 (1H, m), 4.95 (1H, m), 4.35 (1H, m), 4.43 (1H, d), 4.10-3.80 (7H, m), 3.78 (1H, m), 3.68 (1H, m), 3.57 (1H, m), 3.21 (2H, m), 2.81 (2H, m), 2.52 (2H, m), 2.31 (6H, s), 1.98 (2H, m), 1.74-1.63 (5H, m), 1.60-1.45 (6H, m), 1.41-1.30 (5H, m), 1.26 (11H, m), 1.09 (3H, d), 0.95-0.85 (5H, m).

### EXAMPLE 16

¹H NMR (CDCl₃) δ 7.92 (1H, d), 7.86 (1H, d), 7.62 (1H, d), 7.44 (1H, m), 7.35 (1H, m), 6.62 (1H, d), 6.46 (1H, dd), 4.95 (1H, dd), 4.43 (1H, m), 4.34 (1H, d), 3.92-3.73 (6H, m), 3.65 (1H, m), 3.55 (1H, m), 3.39 (2H, m), 3.21 (3H, m), 2.82 (2H, m), 2.51 (2H, m), 2.30 (6H, s), 1.96 (2H, m), 1.70-1.61 (5H, m), 1.53 (6H, s), 1.36 (5H, m), 1.24 (11H, m), 1.08 (3H, d), 0.91 (5H, m).

### EXAMPLE 17

¹H NMR (CDCl₃) δ 7.36 (2H, d), 7.29 (2H, d), 6.70 (1H, dd), 5.72 (1H, d), 5.22 (1H, d), 4.94 (1H, dd), 4.41 (1H, d), 4.33 (1H, d), 3.90-3.75 (6I1, m), 3.64 (2H, m), 3.56 (1H, m), 3.38 (1H, m), 3.20 (2H, m), 2.81 (2H, m), 2.50 (2H, m), 2.30 (6H, s), 1.96 (2H, m), 1.72-1.56 (5H, m), 1.52 (6H, m), 1.36 (6H, m), 1.24 (11H, m), 1.08 (3H, d), 0.91 (511, m).

### EXAMPLE 18

¹³C NMR (CDCl₃) δ 204.9, 169.4, 156.1, 149.6, 137.5, 136.6, 131.5, 129.7, 128.4, 127.0, 121.9, 120.4, 103.5, 83.5, 81.6, 80.0, 76.7, 70.3, 69.6, 65.9, 60.2, 51.1, 51.0, 49.5, 47.0, 42.8, 42.1, 40.2, 38.2, 36.3, 28.2, 27.8, 27.1, 25.1, 22.4, 21.3, 20.1, 19.7, 15.6, 14.8, 14.6, 13.6, 13.1, 11.1, 10.7.

### EXAMPLE 19

¹H NMR (CDCl₃) δ8.09 (2I1, d), 7.48 (2H, d), 4.95 (1H, dd), 4.45-4.34 (5H, m), 3.92-3.78 (7H, m), 3.70 (1H, m), 3.56 (1H, m), 3.39 (1H, m), 3.21 (2H, m), 2.88 (2H, m), 2.53 (2H, m), 2.32 (6H, s), 1.95 (2H, m), 1.73-1.56 (5H, m), 1.53 (6H, m), 1.43-1.36 (6H, m), 1.25 (11H, m), 1.09 (3H, d), 0.95-0.85 (5H, m).

The foregoing is merely illustrative of the invention and is not intended to limit the same to the disclosed compounds. Variations and changes which are obvious to one skilled in the art are intended to be within the scope and nature of the invention which is defined in the appended claims.

What is claimed is:

1. A compound having formula (I) 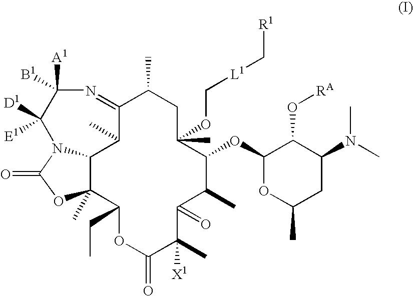
in which,
two of A¹, B¹, D¹, and E¹ are hydrogen, alkyl, alkenyl, alkynyl, cycloalkyl, aryl, heteroaryl, heterocyclyl, —CN, —OH, —SH, —C(O)H, —C(O)R², —C(O)OH, —C(O)OR², —C(O)NR³R⁴, or alkyl substituted with one, two, or three substituents independently selected from the group consisting of —CN, —OH, —SH, halo, aryl, heteroaryl, heterocyclyl, —OR², —SR², —C(O)H, —C(O)R², —C(O)OH, ⁻C(O)OR², —CH═N—OR², —OC(O)R², —OC(O)OR², —C(O)NR³R⁴, —OC(O)NR³R⁴, —NR³R⁴, —N(R⁵)C(O)H, —N(R⁵)C(O)R², —N(R⁵)C(O)NR³R⁴, —N(R⁵)SO₂R², —OR², —SR², —S(O)R², —SO₂R², and —SO₂NR³R⁴, and the remainder are hydrogen; or
A¹ and D¹, A¹ and E¹, B¹ and D¹, or B¹ and D¹ together are one- to five-membered alkylene or two- to five-membered heteroalkylene, and the remainder are hydrogen; or
A¹ and B¹ together are one- to seven-membered alkylene or two- to seven-membered heteroalkylene, and D¹ and E¹ are hydrogen; or
D¹ and E¹ together are one- to seven-membered alkylene or two- to seven-membered heteroalkylene, and A¹ and B¹ are hydrogen;
L¹ is selected from the group consisting of C≡C, (E)—CH═CH, and (Z)—CH═CH;
X¹ is selected from the group consisting of hydrogen and fluoride;
R^{A} is selected from the group consisting of hydrogen and R^{P}, in which R^{P} is a hydroxyl protecting group; and
R¹ is selected from the group consisting of aryl, heteroaryl, and heterocycle;
in which, for the foregoing,
each aryl, heteroaryl, and heterocyclyl is unsubstituted or substituted with one, two, three, four, or five substituents independently selected from the group consisting of alkyl, alkenyl, alkynyl, cycloalkyl, halo, —CN, —OH, —SH, —NH₂, —NO₂, (O), —CF₃, —CH₂CF₃, —CF₂CF₃, —OCF₃, —OCH₂CF₃, —OCF₂CF₃, —OR³⁰, —SR³⁰, —S(O)R³⁵, —SO₂R³⁵, —C(O)H, —C(O)R³⁵, —C(O)OH, —C(O)OR³⁵, —NH(R³⁵), —N(R³⁵)(R³⁵′), —C(O)NH₂, —C(O)NH(R³⁵), —C(O)N(R³⁵)(R³⁶), —OC(O)R³⁵, —OC(O)OR³⁵, —OC(O)NH₂, —OC(O)NH(R³⁵), —OC(O)N(R³⁵)(R³⁶), —NHC(O)H, —NHC(O)R³⁵, —NHC(O)OR³⁵, —NHC(O)NH₂, —NHC(O)NH(R³⁵), —NHC(O)N(R³⁵)(R³⁶), —SO₂NH₂, —SO₂NH(R³⁵), —SO₂N(R³⁵)(R³⁶), R⁴⁰, and alkyl substituted with one or two substituents independently selected from the group consisting of halo, —CN, —OH, —SH, (O), —OR³⁰, —SR³⁰, —C(O)OH, —C(O)OR³⁵, —NH₂, —NH(R³⁵), —N(R³⁵)(R³⁶), —C(O)NH₂, —C(O)NH(R³⁵), —C(O)N(R³⁵)(R³⁶), —OC(O)R³⁵, —OC(O)NH₂, —OC(O)NH(R³⁵), —OC(O)N(R³⁵)(R³⁶), —SO₂NH₂, —SO₂NH(R³⁵), —SO₂N(R³⁵)(R³⁶), and R⁴⁰;
R³⁰ is selected from the group consisting of alkyl and alkyl substituted with a substituent selected from the group consisting of halo and OR⁴⁵;
R³⁵ and R³⁶ are independently selected alkyl;
R⁴⁰ is selected from the group consisting of phenyl, naphthyl, furyl, thienyl, pyrrolyl, oxazolyl, thiazolyl, imidazolyl, pyrazolyl, isoxazolyl, isothiazolyl, 1,2,3-oxadiazolyl, 1,2,3-thiadiazolyl, 1,3,4-thiadiazolyl, 1,2,3-triazolyl, tetrazolyl, pyridyl, pyrazinyl, pyrimidinyl, pyrrolidinyl, inidazolidinyl, piperidinyl, piperazinyl, morpholinyl, or thiomorpholinyl, each of which is unsubstituted or substituted with one, two, or three substituents independently selected from the group consisting of alkyl, alkenyl, alkynyl, cycloalkyl, halo, —CN, —OH, —SH, —NO₂, (O), —CF₃, —CH₂CF₃, —CF₂CF₃, —OCF₃, —OCH₂CF₃, —OCF₂CF₃, —OR⁴⁵, —SR⁴⁵, —S(O)R⁵⁰, —SO₂R⁵⁰, —C(O)H, —C(O)R⁵⁰, —C(O)OH, —C(O)OR⁵⁰, —NH₂, —NH(R⁵⁰), —N(R⁵⁰)(R⁵¹), —C(O)NH₂, —C(O)NH(R⁵⁰), —C(O)N(R⁵⁰)(R⁵¹), —OC(O)R⁵⁰, —OC(O)OR⁵⁰, ⁻OC(O)NH₂, —OC(O)NH(R⁵⁰), —OC(O)N(R⁵⁰) (R⁵¹), —NHC(O)H, —NHC(O)R⁵⁰, —NHC(O)OR⁵⁰, —NHC(O)NH₂, —NHC(O)NH(R⁵⁰), —NHC(O)N(R⁵⁰)(R⁵¹), —SO₂NH₂, —SO₂NH(R⁵⁰), and —SO₂N(R⁵⁰)(R⁵¹);
R⁴⁵ is alkyl;
R⁵⁰ and R⁵¹ are independently selected alkyl.

2. A compound of claim 1 having formula (I) 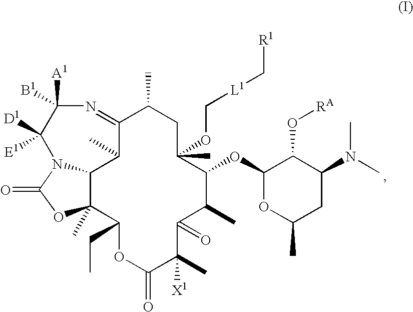
in which
A¹, B¹, D¹, and E¹ are hydrogen;
X¹ is hydrogen;
L¹ is C≡C;
R^{A} is hydrogen;
R¹ is selected from the group consisting of aryl, heteroaryl,
in which the aryl is phenyl and the heteroaryl is pyridyl and quinolinyl, and
in which the foregoing aryl and each foregoing heteroaryl is unsubstituted or substituted with a substituent selected from the group consisting of alkenyl and R⁴⁰,
in which R⁴⁰ is selected from the group consisting of furyl, pyridyl, 1,2,3-thiadiazolyl, thiazolyl, thienyl, and tetrazolyl, each of which is unsubstituted or substituted with one alkyl substituent.

3. A compound of claim 1 having formula (I) 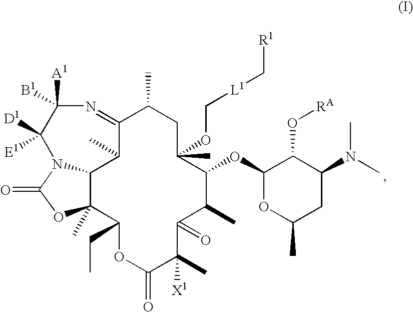
in which
A¹, B¹, D¹, and E¹ are hydrogen;
X¹ is hydrogen;
L¹ is C≡C;
R^{A} is hydrogen;
R¹ is selected from the group consisting of aryl and heteroaryl,
in which the aryl is phenyl and the heteroaryl is pyridyl and quinolinyl, and
in which the foregoing aryl and each foregoing heteroaryl is unsubstituted or substituted with a substituent selected from the group consisting of C₂-alkenyl and R⁴⁰,
in which R⁴⁰ is selected from the group consisting of furyl, pyridyl, 1,2,3-thiadiazolyl, thiazolyl, thienyl, and tetrazolyl, each of which is unsubstituted or substituted with one C₁-alkyl substituent.

4. A composition for prophylaxis or treatment of methicillin-resistant staphylococcus aureus infections in a fish or a mammal, the composition comprising a therapeutically effective amount of a compound of claim 1.

5. A method for prophylaxis and treatment of methicillin-resistant staphylococcus aureus infections in a fish or a mammal comprising administering thereto a therapeutically effective amount of a compound of claim 1.

6. A compound of claim 1 selected from the group consisting of
(3aS,4R,7R,9R,10R,11S,13R,15R,15aR)-4-ethyl-3a,7,9,11,13,15-hexamethyl-2,6,8-trioxo-11-((4-pyridin-2-ylbut-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside,
(3aS,4R,7R,9R,10R,11S,13R,15R,15aR)-4-ethyl-3a,7,9,11,13,15-hexamethyl-2,6,8-trioxo-11-((4-(4-(1,2,3-thiadiazol-5-yl)phenyl)but-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside,
(3aS,4R,7R,9R,10R,11S,13R,15R,15aR)-4-ethyl-3a,7,9,11,13,15-hexamethyl-2,6,8-trioxo-11-((4-quinolin-3-ylbut-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside,
(3aS,4R,7R,9R,10R,11S,13R,15R,15aR)-4-ethyl-3a,7,9,11,13,15-hexamethyl-2,6,8-trioxo-11-((4-(4-thien-2-ylphenyl)but-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside,
(3aS,4R,7R,9R,10R,11S,13R,15R,15aR)-4-ethyl-3a,7,9,11,13,15-hexamethyl-2,6,8-trioxo-11-((4-(4-(1,3-thiazol-2-yl)phenyl)but-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside,
(3aS,4R,7R,9R,10R,11S,13R,15R,15aR)-4-ethyl-11-((4-(4-(2-furyl)phenyl)but-2-ynyl)oxy)-3a,7,9,11,13,15-hexamethyl-2,6,8-trioxododecahydro-14,1-(epiazenoethano)oxacyclotetradecino[4,3-d][1,3]oxazol-10-yl 3,4,6-trideoxy-3-(dimethylamino)-β-D-xylo-hexopyranoside,
(3aS,4R,7R,9R,10R,11S,13R,15R,15aR)-4-ethyl-3a,7,9,11,13,15-hexamethyl-2,6,8-trioxo-11-((4-(4-vinylphenyl)but-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside,
(3aS,4R,7R,9R,10R,11S,13R,15R,15aR)-4-ethyl-3a,7,9,11,13,15-hexamethyl-2,6,8-trioxo-11-((4-(4-pyridin-2-ylphenyl)but-2-ynyl)oxy)dodecahydro-14,1-(epiazenoethano)oxacyclotetradecino[4,3-d][1,3]oxazol-10-yl 3,4,6-trideoxy-3-(dimethylamino)-β-D-xylo-hexopyranoside, and
(3aS,4R,7R,9R,10R,11S,13R,15R,15aR)-4-ethyl-3a,7,9,11,13,15-hexamethyl-11-((4-(4-(2-methyl-2H-tetraazol-5-yl)phenyl)but-2-ynyl)oxy)-2,6,8-trioxododecahydro-14,1-(epiazenoethano)oxacyclotetradecino[4,3-d][1,3]oxazol-10-yl 3,4,6-trideoxy-3-(dimethylamino)-β-D-xylo-hexopyranoside.
